(19) United States
(12) Patent Application Publication
(10) Pub. No.: US 2017/0360295 A1
(43) Pub. Date: Dec. 21, 2017

(54) SYSTEM AND METHOD FOR IMPROVED DISPLAY

(71) Applicant: IMPROVED VISION SYSTEMS (I.V.S.) LTD., Even Yehuda (IL)

(72) Inventors: Dan OZ, Even Yehuda (IL); Michael BELKIN, Givat Shmuel (IL); Ran YAM, Jerusalem (IL); Oren YEHEZKEL, Ramat Gan (IL)

(21) Appl. No.: 15/533,096

(22) PCT Filed: Dec. 22, 2015
(86) PCT No.: PCT/IL2015/051242

§ 371 Date: Jun. 5, 2017

Related U.S. Application Data
(60) Provisional application No. 62/095,350, filed on Dec. 22, 2014.

(51) Int. Cl. A61B 3/113; G06K 9/32; A61B 3/032

(52) U.S. Cl. CPC A61B 3/113; A61B 3/032; G06K 9/3233

(57) ABSTRACT

A system and a method are presented, configured for use in data display. The method comprising: providing data about vision requirement of a user, providing data about content to be displayed to the user and generating and displaying initial display data on a display device, and identifying a region of interest of the user within the display data. The method further comprising processing the display data in accordance with the data about vision requirements of the user for generating refreshed and modified display data comprising suitable image processing for at least a portion of the display data within the region of interest and transmitting the refreshed display data to be displayed on the display device. The refreshed and corrected display data thus provides improved image display to the user within the region of interest.

### TECHNOLOGICAL FIELD

The present invention is in the field of corrected image display for improving user experience in accordance with vision requirements, and specifically relates to improving vision for users having vision impairment.

### BACKGROUND

Out of the five senses that humans have, the sight is the most important and dominant sense. Actually, vision loss is number one health fear. The eye, which is the front end of the vision system, combined with the optical nerve and the brain, form a very complex and delicate system. Because of its complexity, many illnesses affect vision and dramatically reduce the patient quality of life. There are hundreds of millions of people worldwide who suffer from vision related illnesses.

Today, as life expectancy is increasing and the elderly population is rapidly growing, those eye impairments are more significant and there is a need to alleviate the vision quality and thereby the visual quality of life of the elderly.

Eye treatments progressed rapidly during the past 50 years, however, in many cases, the modern medicine world cannot provide cure that will completely fix the visual impairment and in many cases can only slow the pathologic processes and provide very limited remedy for the patients.

Improving the visual capabilities of low vision patients will not only increase their quality of life but also decrease the burden on the society.

With technological development, increasing number of people find the need to use various display systems for information consumption. Typically, such information may be textual and/or pictorial in nature and may relate to professional or entertaining needs.

Currently available image displaying devices provide a selection of accessibility tools providing users the ability to vary certain properties of display data to compensate for vision requirements.

### GENERAL DESCRIPTION

There is a need in the art for a novel technique for increasing display accessibility and enhancing user experience with respect to display data. The technique of the present invention enables online tailored display of selected content in accordance with vision requirements of the user while updating display characteristics based on regions of user interest within the displayed content.

The tailored display generally includes a region of the display that is corrected in accordance with the defined vision requirements, while the display data around the corrected region may remain as originally defined (by the content or display preferences). The corrected region is determined based on determined region of interest of the user within the display data. Thus the corrected region may follow the user's attention while reading text, viewing photos or videos, and apply correction to at least a portion of the display data based on user's attention.

Generally, the technique is based on predetermined vision requirements, which may be associated with known vision impairments of the user. Once the vision impairments of a user are determined (diagnosed professionally or self-diagnosed by the user or determined using one or more automated techniques), proper image processing actions can be engaged, enabling to enhance user's experience by providing displayed content that is appropriate for the user. For example, if the user is diagnosed with certain vision distortion, proper image processing may include distortion reversing. Alternative image processing actions may include enlargement or isolation of a portion of the data, increasing contrast or varying brightness as well as additional image processing actions as will be described further below. Generally, the image correction may be uniform correction, i.e. correction function independent of coordinates within the corrected region, or non-uniform correction such that the correction function is a function of coordinates within the corrected region.

Generally, the technique of the invention generally comprises providing data about vision requirement of a user. This data may be pre-stored in a corresponding storage sector or determined by the user. Additionally, data about content to be displayed is provided/selected and appropriate initial display data is generated to be displayed to the user on a display device. The content may generally be any type of displayed content such as area views, written documents, internet sites, data sheets, pictures, video etc. The content is typically updated in accordance with user interaction such as selection of additional content or different portions of the content to be displayed. It should also be noted that the terms content and display data as described here should be interpreted broadly and relate to any data displayed on a display device including system operational data and operating system interface as well as data selected by the user or any third party for display.

Further, a region of interest of the user within the display data is to be identified. The region of interest (ROI) is typically a portion of the display data including one or more data elements forming together an information piece. The selection of the ROI in accordance with size, shape and location typically provides a portion of the display data enabling the user to better see the information piece(s) and therefore understand or grasp the information therein. For example, such information piece(s) may be a single word, several words within a sentence, line or a portion of an image having size and shape that may be associated with contours of the image, or any part of the screen including, e.g., system interface data.

The user's ROI within the display data may be determined based on line of sight of the user, indicating a point in the display region where one or more of the user's eyes are directed at. The line of sight may be determined by eye tracking, pointing device (e.g. mouse) or in accordance with calculated or anticipated patterns of user's attention. In some configurations, the ROI may be determined based on movement history or movement characteristics of the line of sight within a predetermined time period, this is to compensate for fast or unwanted movements of the user's line of sight that may be unrelated to the ROI. In some other configurations, such fast movements of the line of sight may be compensated by appropriate image processing actions, for example for providing image correction associated with Nystagmus of one or both of the user's eyes.

At least a portion of the display data, associated with the ROI of the user undergoes one or more image processing actions selected in accordance with the vision requirements. The image processing actions may generally include one or more filters or image processing operations selected to improve visibility of the information piece(s) within the ROI thereby providing improved visual performance for the user. For example, if the vision requirements of the user relate to increased separation between words in text, or the use of large font, the image processing actions may include selection of the textual information within the ROI, and generating display data with the selected textual information enlarged and/or with increased spacing, presented/displayed over the original image data. Based on the image processing action, refreshed display data may be generated and transmitted to the display device.

The display data may be refreshed periodically, e.g. in accordance with refresh rate of the display device in use, or in accordance with refresh rate of the content being displayed, while the ROI of the user may vary with time, thereby providing the user with continuously improved display data tailored to the personal vision requirements.

In this context it should be noted that the present technique may be implemented by a system, e.g. computer system, connectable to one or more display devices such as screen, projector unit or multiple screens, which may be head-mounted acting as glasses with display. In some embodiments, the display device may comprise separate display for each of the user's eyes either by separate screens, or by streaming different images to each eye separately from a single screen allowing tailoring of the image processing actions and of the display data to different vision requirements of each of the user's eye. Generally, such separate displays to the two eyes may be used to provide three dimensional experiences, however as described herein below, the present technique may provide, additionally or alternatively, an improved image display suitable for vision requirement that may vary between the user's eye.

Further, the system may be connectable to one or more input devices providing indication associated with line of sight of the user. Such input devices may include pointing devices (e.g. mouse, keyboard, tracking pencil etc.) or input devices capable of providing input indicative of actual line of sight of the user based on orientation of one or both of the user's eyes. For example, the technique of the invention may utilize eye tracking unit providing data about orientation of one or both the user's eyes or a camera unit providing image data of the user. Such image data may be processed to provide data about the line of sight based on location and orientation of one or both of the user's eyes.

Thus, according to one broad aspect of the invention, there is a provided method for use in data display comprising: providing data about vision requirement of a user, providing data about content to be displayed to said user and generating and displaying initial display data on a display device, and identifying a region of interest of the user within the display data; and processing the display data in accordance with said data about vision requirement of the user for generating refreshed display data comprising suitable image processing for at least a portion of the display data within the region of interest and transmitting said refreshed display data to be displayed on the display device; thereby providing said user with improved image display within the region of interest.

Identifying the ROI of the user may comprise processing said content to be displayed and determining one or more data portions based on said content. Identifying the ROI of the user may also comprise providing data about a line of sight of at least one eye of said user to thereby identify said ROI. More specifically, identifying of the region of interest of the user may comprises utilizing history data of the line of sight within a predetermined time period and selecting variations of the line of sight corresponding to said display data.

Generally providing data about line of sight of the user may comprise determining a point within the display region associated with the estimated location of user attention.

According to some embodiments, providing data about line of sight of the user may comprise receiving data about movement history of a pointing device (e.g. mouse) and processing said data about movement history for determining said data about line of sight of the user.

According to some embodiments, providing data about line of sight of the user may comprise providing data about movement of at least one of the user's eyes and comparing said data with predetermined location on the display device, thereby determining a location within said display data being within line of sight of the user.

Providing data about movement of at least one of the user's eyes may comprise: collecting image data of the user from an imager mounted at a predetermined location with respect to said display device and processing said image data to determine orientation of at least one of the user's eyes with respect to said display device.

For example, providing data about line of sight of the user may comprise providing data about individual movement of each of the user's eyes and determining a right eye line of sight and a left eye line of sight. The display data may also comprise right-eye display data and left-eye display data and processing of the display data comprises applying first and second image processing to at least a portion of said right-eye display data and left-eye display data respectively. Such image processing for at least a portion of the right-eye display data and left-eye display data may be similar or different.

Said providing data about movement of at least one of the user's eyes may further comprise determining if said line of sight of at least one of the user's eyes is within region of display or outside of said region of the display and holding update of said region of interest for at least one of the user's eyes when line of sight of said eye being outside of the display region.

Additionally or alternatively, said providing data about movement of at least one of the user's eyes may further comprise determining if said line of sight of at least one of the user's eyes is within region of display or outside of said region of the display and transmitting command to darken the corresponding display device (e.g. by blocking the eye channel by different means such as a spectacle mounted shutter) with respect to said at least one of the user's eye being directed outside of said region of the display.

According to some embodiments, said applying said image processing to at least a portion of the display data within the Region of Interest (ROI) of the user may comprise applying at least one of the following filters in accordance with said data about vision requirement of the user: image shift, image rotation, image distortion reversing, image magnification, increase in spacing between image portions (e.g. words), apply blur to surrounding of said ROI, emphasize portion of the ROI, isolate portion of the ROI, increase contrast within said ROI, decrease contrast of surrounding, increase or decrease brightness of ROI, replace image colors within the ROI with preselected color map, remove defected portions of the display data, mark contours within the display data and mark pattern edges of in the display data.

According to some embodiments, said providing data about content to be displayed, determining a ROI of the user, applying a corresponding filter to at least a portion of the display data within the ROI of the user, generating a refreshed display data and transmitting said display data to a display device are typically performed continuously, thereby enabling continuous vision improving display data to the user.

According to some embodiments, the technique may further comprise receiving and generating audio signals associated with said display data.

According to some embodiments, the method further comprising determining one or more command regions within the display data and determining if said region of interest of the user within the display data being within said one or more command regions to thereby activate one or more predetermined commands.

According to some embodiments, the method further comprising providing eye-tracking data and determining one or more gestures corresponding to eye movement or other eye behaviors of the user, said one or more gestures being associated with one or more predetermined commands to thereby activate said one or more predetermined commands.

The one or more predetermined commands may comprise one or more commands associated with selection of said data to be displayed or commands associated with selection of vision requirement of the user thereby affecting selection of a image processing to be applied to said at least a portion of the display data.

According to some embodiments, the method may be configured for providing reading assistance, comprising processing the display data and determining one or more data portions to be highlighted, determining a progress rate for reading and highlighting selected data portions accordingly. The method may further comprise determining line of sight of the user and controlling guided reading progress in accordance with variation in said line of sight; said controlling comprises at least one of the following actions: holding reading progress, resume reading progress from previous location, resume reading progress from a selected location, vary reading speed. Such method may also comprise determining a distance between line of sight of the user and current highlighted selected data portion, and if said distance exceeds a predetermined threshold, updating eye tracking calibration data to align line of sight with highlighted data portions.

Generally, the above describe method may be implemented as computer program code means for performing the above described steps when said program is run on a computer.

According to one other broad aspect of the invention there is provided a program storage device readable by machine, tangibly embodying a program of instructions executable by the machine to perform a method for use in data display comprising: providing data about vision requirement of a user,

providing data about content to be displayed to said user and generating and displaying initial display data on a display device, and

identifying a region of interest of the user within the display data;

processing the display data in accordance with said data about vision requirement of the user for generating refreshed display data comprising suitable image processing for at least a portion of the display data within the region of interest and transmitting said refreshed display data to be displayed on the display device; thereby providing said user with improved image display within the region of interest

According to yet another broad aspect of the invention, there is provided a system for providing improved display data comprising: at least one processing unit, input and output module and a memory utility, and connectable to at least one display device; the system is configured and operable for generating improved display data in accordance with predetermined data about vision requirement of the user; the processing unit comprises:

display data generator configured and operable for receiving data to be displayed and generating and transmitting display data to a display device;

region of interest (ROI) identifier configured and operable for receiving said display data and determining a region of interest (ROI) within said display data; and

local image processing module configured and operable for receiving said predetermined data about vision requirement of the user and for processing at least a portion of the display data in accordance with said ROI of user within the display data for applying one or more corresponding image processing and for transmitting a resulting improved vision data to the display data generator for providing refreshed display data to the user.

The processing unit may further comprise line of sight detector configured and operable for determining line of sight of the user; said ROI identifier is configured and operable for determining a region of interest (ROI) in accordance with data about line of sight received from the line of sight detector.

The line of sight detector may be configured and operable for determining said line of sight of the user in accordance with movement history of a pointing device connected to said input module of the system.

The system may be connectable with an eye tracking unit, said line of sight detector being configured and operable for receiving data about at least one of location and orientation of at least one of the user's eyes to thereby determine said line of sight of the user.

The line of sight detector may be configured and operable for receiving data about individual movement of each of the user's eyes and determining right eye line of sight and left eye line of sight.

The display data generator may be configured for generating and transmitting display data comprising right-eye display data and left-eye display data to be appropriately displayed to the user for providing seamless individual display for each of the user's eyes. Further, the local image processing module may be configured and operable for selectively applying first and second filters to a portion of the right-eye display data and left-eye display data respectively in accordance with said right eye line of sight and left eye line of sight and said data about vision condition of the user.

According to some embodiments, the line of sight detector is further configured for determining if said line of sight of at least one of the user's eye is within region of the display or outside of said region of the display and generating corresponding notification to the ROI identifier, said ROI identifier is further configured and operable for distinguishing between willing or unwilling shift in the line of sight and determines corresponding variation in the ROI of the user.

The local image processing module may be further configured and operable for removing at least a portion of the image data in accordance with a notification about unwilling shift in the line of sight from the ROI identifier, thereby darkening at least a portion of the display data is response to large shift in line of sight or blocking the eye channel by different means such as a spectacle mounted shutter.

According to some embodiments, the local image processing module being configured and operable for applying at least one of the following image processing in accordance with said data about pre-existing vision condition of the user: image shift, image rotation, image distortion reversing, image magnification, increase in spacing between image portions (e.g. words), apply blur to surrounding of said ORI, emphasize portion of the ROI, isolate portion of the ROI, increase contrast within said ROI, decrease contrast of surrounding, increase or decrease brightness of ROI, replace image colors within the ROI with preselected color map, remove defected portions of the display data, mark contours within the display data and mark pattern edges of in the display.

In some configurations, the system may be configured for continuously refreshing said display data in accordance with varying line of sight of the user and in accordance with variation in said data to be displayed.

According to some embodiments, said processing unit may further comprise a local command generator configured and operable for determining one or more command regions within the display data and for generating a corresponding one or more predetermined commands in response to user line of sight being detected within said one or more command regions, said line of sight detector is further configured for generating notification to the command generator when line of sight is determined to be within said one or more command regions to thereby activate one or more predetermined commands

According to some embodiments, said processing unit may further comprise a gesture command generator configured and operable for determining one or more command associated with gestures of eye movement or other eye behaviors, the system being connectable to an eye tracking unit configured for providing eye-tracking data, said gesture command generator is further configured for receiving said eye tracking data and identifying one or more gestures with said eye tracking data to thereby activate said one or more predetermined commands associated with an identified gesture.

The one or more predetermined commands may comprise one or more commands associated with selection of said data to be displayed or one or more commands associated with selection of a filter to be applied to said at least a portion of the display data.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates a system according to some embodiments of the present invention;

FIG. 2 illustrates a process according to some embodiments of the present invention, in a way of a block diagram;

FIG. 3 illustrates in a way of a block diagram a method for identifying a region of interest of the user according to some embodiments of the invention;

FIG. 4 illustrates a system for use in data display utilizing local/gesture command generator according to some embodiments of the invention;

FIG. 5 exemplifies image correction for users with diplopia according to some embodiments of the invention;

FIG. 6 exemplifies image correction for users with Nystagmus or oscillopsia according to some embodiments of the invention;

FIG. 7 exemplifies image correction for users with tunnel vision, scotoma or reduced central visual ability according to some embodiments of the invention;

FIG. 8 exemplifies image correction for users with hemianopia or damaged visual field loss according to some embodiments of the invention;

FIGS. 9A and 9B exemplify image correction for users with visual distortion according to some embodiments of the invention; FIG. 9A exemplifies reverse distortion correction and FIG. 9B exemplifies variation in ROI location and corresponding change in image processing;

FIGS. 10A and 10D illustrate reading assistance according to some embodiments of the invention, FIG. 10A exemplifies text enlargement, FIG. 10B, FIGS. 10C and 10D exemplifies reduction of crowding;

FIG. 11 illustrates auto eye tracking dynamic working distance error compensation according to some embodiments of the invention; and

FIGS. 12A and 12B exemplify the use of guided reading, text highlighting, and eye tracking dynamic calibration according to some embodiments of the invention.

### DETAILED DESCRIPTION OF EMBODIMENTS

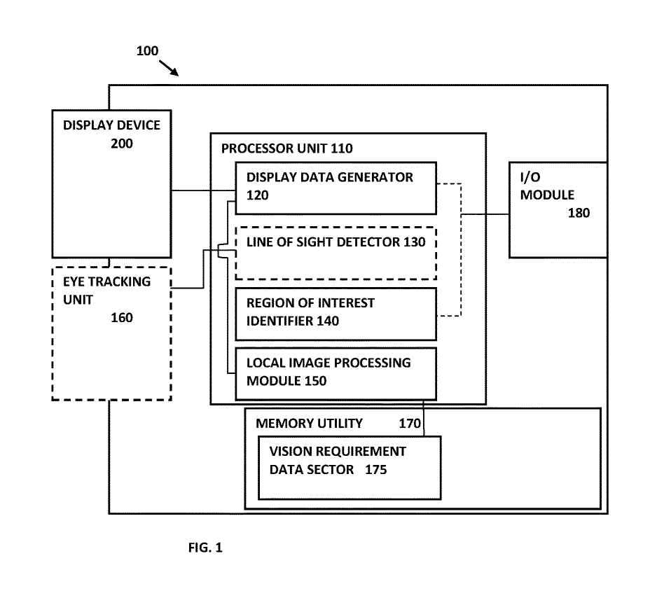

As indicated above, the present invention provides a system and method of use in data display for providing improved vision experience in accordance with user vision requirements, typically associated with vision impairment. Reference is made to FIG. 1 schematically illustrating a computerized system 100 for providing improved display according to some embodiments of the invention. The system 100 includes at least one processing unit 110, memory utility 170 configured for storing data and input/output module 180 connectable to one or more input or output units. The system 100 is further connectable to one or more display devices 200 and may also be connectable to an eye tracking unit 160 for providing eye tracking data. The system is configured and operable for generating improved display data in accordance with predetermined data about vision requirement of the user, e.g. based on data stored in vision requirement data sector 175 of the memory utility.

Generally, the system 100 is configured to provide display data based on content, selected by the user and stored in the memory utility 170 or received through external connection (e.g. through the input/output module 180), and transmit the display data to the one or more display devices 200 to be presented to the user. The data is displayed while at least a portion of the image data is processed in accordance with the user's vision requirements, for instance to enable users with difficulties in vision (e.g. vision impairment) to properly see the data.

To this end the processing unit 110 includes display data generator 120 configured and operable for generating display data based on content to be displayed with or without image processing, and for transmitting the display data to a display device 200; region of interest (ROI) identifier 140 configured and operable determining a region of interest (ROI) of the user within the display data; and local image processing module 150 configured and operable for applying selected one or more image processing actions on at least a portion of the display data associated with the determined ROI, and transmitting refreshed display data to the display data generator 120 for refreshing the display data.

The image processing actions are selected in accordance with the vision requirements of the user in order to provide the user with improved vision within the ROI. For example, and as will be described in more details further below, the local image processing module 150 may be configured to utilize at least one of uniform or non-uniform local image processing actions selected to provide the user's vision requirements.

To this end uniform image processing corresponds to processing of at least a portion of the display data by applying a function that is independent of coordinates within the determined ROI of the user. For example, uniform processing actions may include one or more of the following: shifting of image portion, rotation of image portion, magnification of the image within the ROI, blurring of surrounding of the ROI, brightness or contrast increase within the ROI or decrease in surrounding thereof, replacing color map within the ROI, enhancing contours and pattern edges within the ROI and additional selected image processing actions.

This is while a non-uniform image processing relates to processing as a function of coordinates within the ROI of the user. Such non-uniform image processing function may include one or more of the following: distortion reversing filter, variation (e.g. increase) spacing between image portions (e.g. words), emphasizing portion of the ROI, isolating portion of the ROI from surrounding details, removing defected portions of the display data, applying non uniform magnification, applying non uniform brightness level correction, and various other image processing algorithms.

Generally, the present technique provides users having certain vision impairments with tailored improved display data. A user may be diagnosed by a professional such as ophthalmologist to map his vision disabilities, or be self-diagnosed or diagnosed by any other person or automatic system capable of mapping vision difficulties. It should be noted that certain image processing actions provided by the present technique may require high-level diagnosis of the user's vision and accurate mapping of retina sensitivity and image perception to provide meaningful corrected display data. This is while certain other image processing actions are relatively simple and may be used effectively based on limited diagnosis, e.g. self-diagnosis.

The local image processing module (LIPM) 150 is configured and operable for receiving the data about vision requirement of the user and for applying selected one or more image processing action on at least a portion of the display data. The portion of the display data undergoes image processing is generally selected in accordance with the ROI of user as determined by the ROI identifier 140, however the image processing may extend outside of the ROI to provide seamless boundaries to smooth the resulting display data as the case may be. The LIPM 150 provides the display data generator 120 with refreshed or corrected display data for updating the display device.

Generally, as the content to be displayed and the display data, as well as the ROI of the user within the display data, may change with time, the processing unit 110 may typically be configured to operate in continuous manner and update the display data in a predetermined refresh rate. Generally, the refresh rate may be determined based on frame rate of the displayed content. In some configurations the refresh rate may be determined based on system capabilities and/or user preferences.

As also shown in FIG. 1, the processing unit 110 may, in some embodiments further include a line of sight detector 130. The line of sight detector 130 is configured and operable for determining line of sight of the user, i.e. one or two points within the display region, at which the user's eyes are directed respectively. The line of sight detector transmits data about the line of sight of the user to the ROI identifier 140 for determining the region of interest (ROI) in accordance therewith.

The line of sight detector 130 may be configured to determine the line of sight utilizing one or more techniques and different types of data input. In some embodiments, the line of sight detector 130 may be configured to communicate with one or more input devices such as pointing device (e.g. mouse, pen, touch sensitive regions of the screen, keyboard etc.). The line of sight detector may be configured for processing the input data in accordance with the displayed content, user preferences, user content and ROI history and history of movement of the pointing input data within a predetermined (relatively short) time period to determine one or more points within the display region that correlate with the line of sight of the user.

In some embodiments the line of sight detector 130 is connectable to an input device configured to provide eye tracking data of the user. Such input device may be an eye tracking unit 160 being an integral part of the system 100 or an external unit connectable to the system through the input/output module 180. Generally, the eye tracking unit may be positioned at a static location or attached to a head-mounted unit moving with the user. In some configurations the input device may be a camera unit providing image stream of the user to allow detection of location and orientation of the user's eyes. The processing unit 110 or the line of sight detector 130 thereof may be configured for processing the input image stream to detect location and orientation of at least one of the user's eyes and utilizes data about the user relative location to the display device to determine line of sight of the user.

Generally, the ROI identifier 140 may utilize processing of line of sight history to determine the ROI and adjust it to user's expectations. For example, in case of textual content reading, the user's line of sight is expected to move across lines at a relatively uniform speed; the ROI may be determined as the entire line or a few words and may be refreshed based on the estimation of uniform rate of reading while considering and relating to variations such as jumping back a few lines or words, or line of sight variations associated with vision requirements (e.g. in case of Nystagmus).

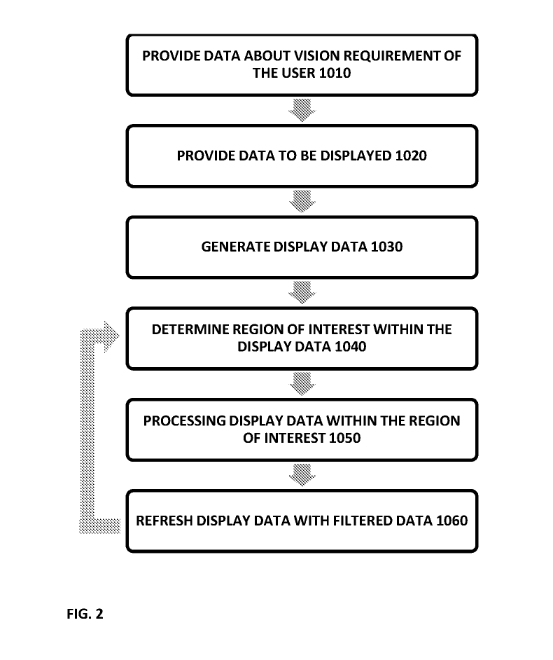

Reference is made to FIG. 2, illustrating in a way of block diagram a method for use in data display according to some embodiments of the invention. In order to provide self-tailored display, data about vision requirement is provided 1010. As indicated above, the data may be based on professional diagnosis or any type of self-diagnosis. Additionally, user selection, or operator selection, of content or data to be displayed is provided 1020 to provide initial display on a display device 1030.

Once there is visible content on the display device, the method is based on determining region of interest (ROI) of the user within the display region 1040. Based on the ROI of the user, the technique of the invention includes processing of at least a portion of the display data within the region of interest 1050. Further, the display data is refreshed 1060 with the newly processed data to provide improved display to the user.

As indicated above, the processing of at least a portion of the display data includes one or more image processing actions selected in accordance with the data about vision requirement. Additionally, the image processing actions may also be selected in accordance with the content type. More specifically, textual data may typically be processed differently than image data.

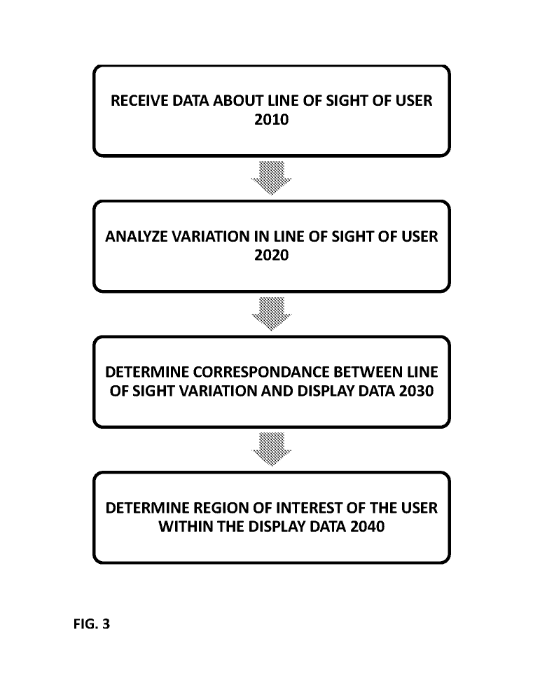

As also indicated above, the technique may include several methods for determining the ROI of the user within the display data. FIG. 3 illustrates in a way of block diagram exemplary method for determining the ROI. The method includes receiving data about line of sight of the user 2010. The data may be received from an eye tracking unit or module or extrapolated from movement history of a pointing device (e.g. mouse). As the line of sight may vary willingly or unwillingly from different reasons, the method may generally include certain processing of line of sight variation 2020. This is particularly important when the line of sight is determined based on input from pointing devices as most users do not hold the pointing mouse located on the exact location on the screen as the content they are currently looking at. Thus, the analysis may generally include analysis of the location variation of the pointing device in accordance with type of content on the display as well as time and user preferences and behavior. In this connection it should be clear that although pointing a device may be used to estimate line of sight and therefore ROI of the user, such estimation may typically require analysis of pointing history. Further, pointing device movement typically includes limited data about vision impairment condition of the user and therefore may typically be suitable where uniform image correction is applied. This is while the use of eye tracking data may provide direct indication of the line of sight of the user, as well as indication about the user's vision impairment, and allow image correction in accordance with current vision impairment of the user. Further, the use of eye-tracking may eliminate or at least significantly reduce the need for hand to eye coordination which may especially be difficult for the elderly population.

In some embodiments, where the line of sight is determined using eye tracking input, utilizing eye tracking unit or input in form of image-stream indicative of the user and including data about the location and orientation of the user's eyes, and analysis thereof to determine line of sight and corresponding one or two points on the display device within the line of sight, may highly simplify the analysis of line of sight history. In such embodiments, the analysis is generally aimed at identifying if the line of sight of the user changes as a result of willing changes of the ROI or not and if the change is indeed associated with the vision requirements of the user.

For example, certain users may suffer from uncontrollable movement of one or both eyes, e.g. suffering from Nystagmus or Nystagmus-like condition. For such users the data about vision requirements may include indication of Nystagmus and possibly data about rate and nature of movement. The corresponding image processing may be a synchronized shift in location of a portion of the display data, either the entire display data or shift corresponding to movement of the eyes, or separate movement of each eye, when separate displays or three-dimensional (3D) type display is used providing separate images to the different eyes. In this case, the ROI identifier may receive data about line of sight and analyze it to determine if the eye movement is associated with the user's condition or is a result of shift in user's attention. If the eye movement is considered as associated with user condition, the ROI is changing as to compensate for the user condition only, to coincide with the line of sight. However, if the eye's movement is determined to be associated with variation in point of attention of the user, the ROI is to be updated according to the superimposed changes of the user attention and the involuntary movement caused by his condition.

In both cases, other suitable image processing action(s) (e.g. shift or rotation) will apply regardless of the ROI movement is caused by voluntary or involuntary change of the user line of sight.

In addition to locating the ROI, the size and shape of the ROI may vary in accordance with the displayed content. To this end the technique may include an analysis step of comparing the line of sight with the displayed content around it 2030. In this connection, as indicated above, the type of content may determine the ROI. For example, in case of textual content, the ROI may be a line of text, two lines of text, or only one or two words in accordance with vision requirements of the user. Further, if the display data is image-type data, the ROI may be defined as a region of certain area around the line of sight. The area itself may be determined 2040 based on vision requirements and/or lines or contours of the image.

As also indicated above, it should be noted that the technique of the invention may be used in two separate screens or in 3D type display devices configured to provide separate display data to each of the user's eyes. In this context, the data about vision requirement may include data about right-eye vision requirements and data about left-eye vision requirements. Additionally, the technique may include identifying right-eye ROI and left-eye ROI separately, e.g. based on right-eye line of sight and left-eye line of sight using eye tracking data. Similarly, the image processing actions may differ between the right and left eye display data in accordance with diagnosed vision requirement that may vary between the eyes.

It should also be noted that the use of right- and left-eye image processing and tracking according to the present technique may be further used for improving user experience regardless of vision requirements. More specifically, in some configurations and some vision requirements, the system may operate to shift or rotate at least a portion of the display data for one of the user's eyes with respect to the other eye. This may be used to compensate for individual unwilling movements of the eyes with respect to each other. However, if one of the eyes moves and is directed outside of the display region, no image shift may be used to equalize the display data. If the line of sight of one eye is detected to be outside of the display region, the LIPM 150 (in FIG. 1) may operate to hold update of the display data for the corresponding eye. Additionally, in some embodiments, where the display device is head mount display device or 3D type display using active shutter glasses, the system 100 may also provide the display device instruction to shut of all light input to the corresponding eye to thereby enable the user to focus on input data to his other eye and proceed with reading, viewing etc.

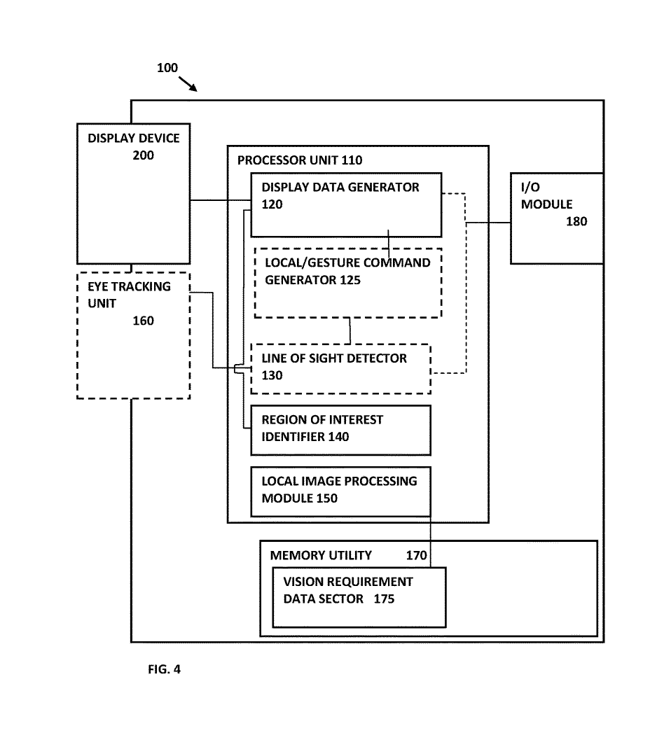

According to some embodiments, the technique of the present invention may also be used to provide certain level of hand free control of system operation. Reference is made to FIG. 4 illustrating a system 100 for use in display of data according to some embodiments of the invention. The system is substantially similar to that shown in FIG. 1, but the processing unit further includes a local/gesture command generator 125. The local/gesture command generator (LG command generator) 125 is configured and operable for determining one or more commands and associating the one or more commands with regions of the display and/or gestures associated with eye movements or other eye behaviors of the user. Thus, once the user aligns his line of sight with one of the defined commands regions in the display, the line of sight detector 130 provides proper indication to the LG command generator 125 that a command has been received. The LG command generator 125 may then operate to activate the command by notifying the processing unit 110 or any module thereof in accordance with the nature of the command Similarly, for gesture type commands, the line of sight detector 130 may identify in line of sight history, movement data associated with a gesture that is defined as command related, to inform the LG command generator 125 and activate the command.

Generally, the system may include a set of predetermined commands; however, such commands may be defined by the user. Typical commands may include commands associated with content to be displayed such as page forwards or backwards, activation of links to further web pages etc. Alternatively, or additionally, such commands may be associated with vision requirement of the user and affect the image processing actions performed by the system. For example, such commands may include further enlargement of display data, increase or decrease in specific processing parameters, switching between user profiles having different vision requirements or shift between 3D type display having separate vision requirements to the different eyes and 2D type display having single image processing for both eyes etc. For example, one or more regions within the boundaries of the ROI may be used as command regions associated with scrolling of the ROI up, down, right or left. This allows the user to shift the displayed image portions at will while working hand-free.

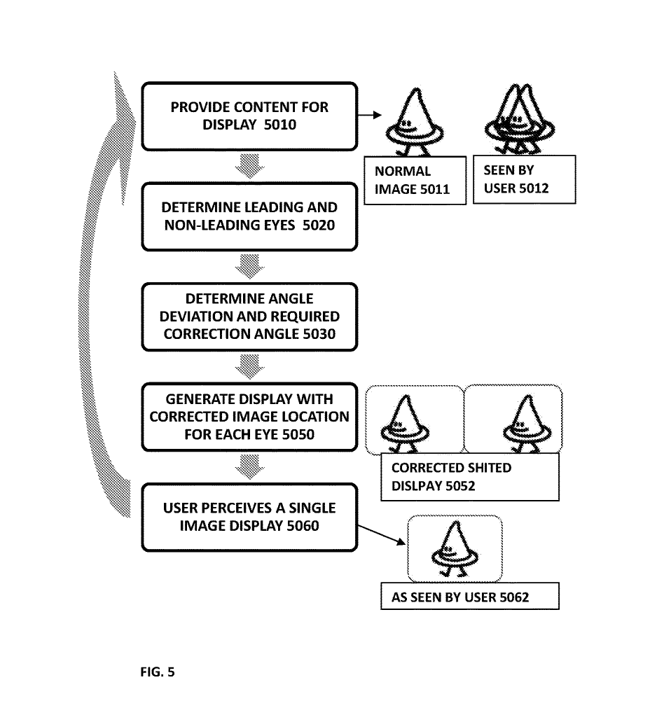

Reference is made to FIGS. 5-9 and FIGS. 10A and 10B exemplifying display corrections according to some embodiments of the invention for users with predefined vision requirements.

In this context FIG. 5 exemplifies display correction for users with Diplopia (Double Vision). This type of correction may typically be used with separate screens or 3D type display capable of providing each of the user's eyes with separate display data. As shown, the original image data is selected 5010, the raw image is shown at 5011 and typically perceived by the user as shown in 5012. In order to provide suitable corrected display, the technique may include selection of the leading eye over the non-leading eye 5020. This may be based on line of sight variations, where the leading eye is relatively stable, however data about leading eye may be provided with vision requirements. Having detected the leading eye and line of sight, the technique includes detection of the angle deviations and required correction 5030 and generates corrected display data 5050 including right-eye display data and left-eye display data with the appropriate corrections 5052. The corrected display data is transmitted to be displayed to the user 5060 to provide corrected display as perceived by user 5062.

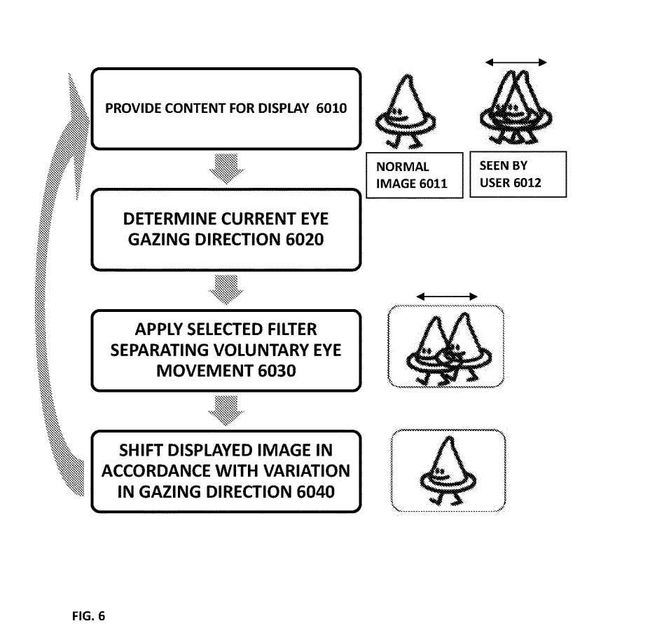

Additionally, FIG. 6 exemplifies correction of display data for users having Nystagmus or Oscillopsia conditions. Generally, in most cases of Nystagmus/Oscillopsia, the involuntary eyes' movements are synchronized between eyes, and therefore the technique may be used with or without 3D-like display device. The technique includes providing of content to be displayed 6010, an example of image content 6011 and how such image is perceived by the user 6012 are also shown. Typically, the image perception of the user may vary based on personal condition, age and state of the patient condition. Generally, people having congenital Nystagmus will perceive a single image but may have difficulty in identifying fine features (low vision acuity). This is while Nystagmus onset at later age may cause greater reduction in vision quality and the patient may feel as if the world is moving around him. Correct and synchronized shift in image display provides image stabilization on the retina and can provide the user with improved data, reducing dizziness and enabling the user to perceive higher image quality (better image acuity). Additionally, the line of sight, or gaze direction of the user is determined 6020 and appropriate filter may be applied to the display data 6030 to compensate for the eye movements. The resulting display data shifts together with the eye movement of the user providing synchronized, corrected and retina stabilized image 6040 to the user.

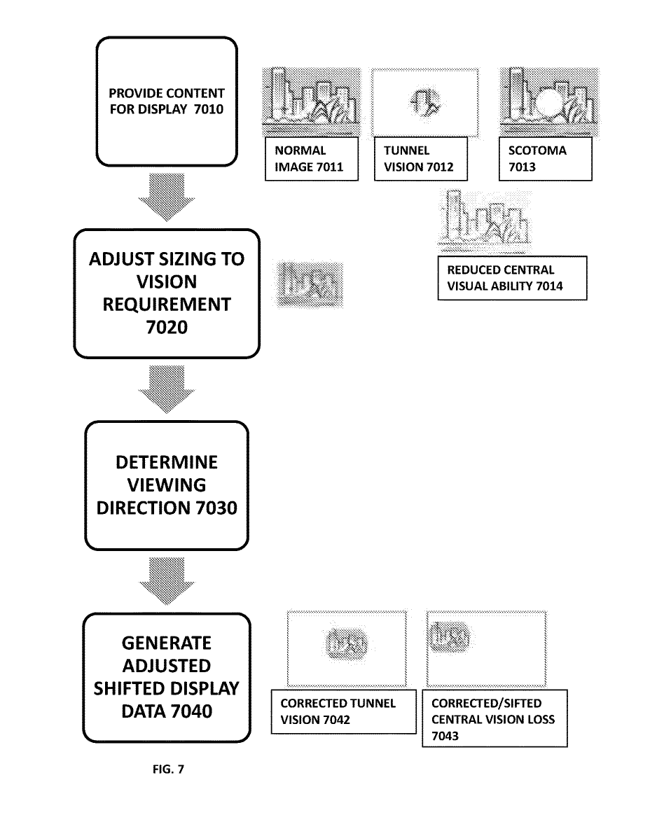

Correction of several other visual defects is exemplified in FIG. 7, relating mostly to visual field defects and correction of central visual acuity reduction.

Generally, data to be displayed is provided 7010, an example of the normal image 7011 and the way this image is perceived by users with visual field defects 7012, scotoma 7013 and reduce central visual ability 7014 are also shown. The image processing actions may generally include adjusting of image sizing 7020 according to the vision requirements, and determining viewing angles 7030 to generate the corrected image 7040. Corrected display data for users with tunnel vision 7042 and central vision loss 7043 are exemplified. Such corrections may typically include resizing of the image, brightness modification and/or shifting of image location with respect to line of sight.

Additional image processing step that are not specifically shown here may typically include image magnification/reduction, which may or may not be in accordance with a linear function (e.g. it can impose high resolution image in the image center and low resolution on the peripheral zones or other functions). In case of a user suffering from profound peripheral vision loss (i.e. Tunnel Vision), the displayed image may be reduced to impose the image in the macula region of the retina, this may increase the field of view (FOV) of the image while lowering resolution.

As also shown, for users with central vision loss (Central Scotoma) or multiple scotomata, proper image correction may include adjusting the image size and applying shift to the image with respect to line of sight to provide image generation on a better location of the retina. This allows the user to see images just in front of him and assist him in development of a Preferred Retinal Location (PRL) and getting used to use the PRL.

Further, correction of central visual acuity may include enlargement of the image size and/or manipulating the image in other ways as described above. The corrected image may be displayed on the display device so as to be imposed on the macula region or any other healthy location of the retina. This allows the user to see better details of the images in front of him and assist him, if required, in development of a PRL.

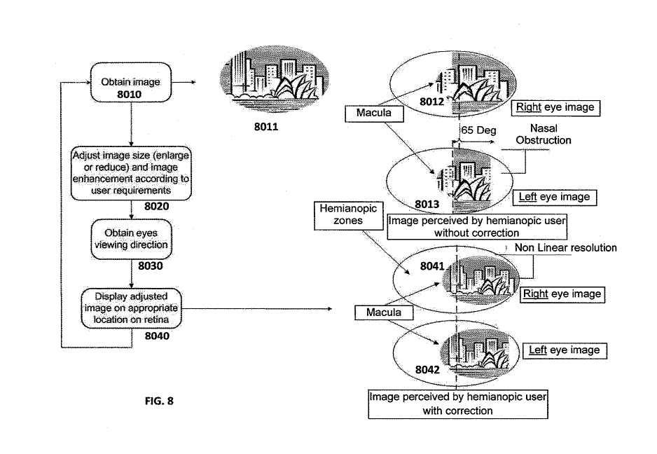

FIG. 8 exemplifies display correction for Hemianopia or half visual field loss. As the image data is provided 8010 and displayed 8011, the user's right and left eyes can “see” the image with several deficiencies as shown in 8012 and 8013. Thus, the technique typically includes adjusting of the image size 8020 according to vision requirements for the user. The image magnification or size reduction may or may not be a linear function (e.g., it can impose high resolution image in the image center and low resolution on the peripheral zones or other functions). It should be noted that the corrected image portions may overlap as for many patients, macular zones may remain intact. As the eye's viewing directions are obtained 8030, corrected display data can be generated 8040 and displayed in accordance with eye directions to form the image data on healthy regions of the user's retina. Examples of the corrected display for the right and left eyes are shown in 8041 and 8042 Similarly, by displaying the image data to be projected to healthy regions of the user's retina, the technique may be also used to assist the user in development of PRL.

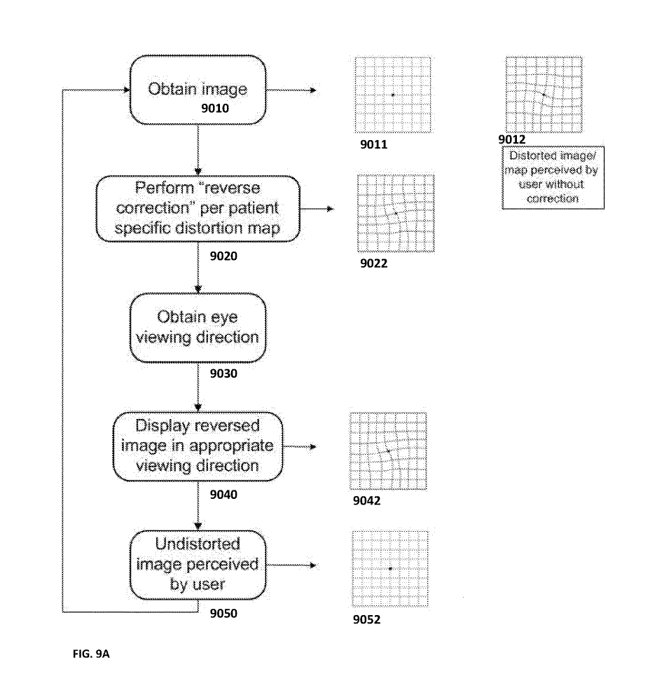

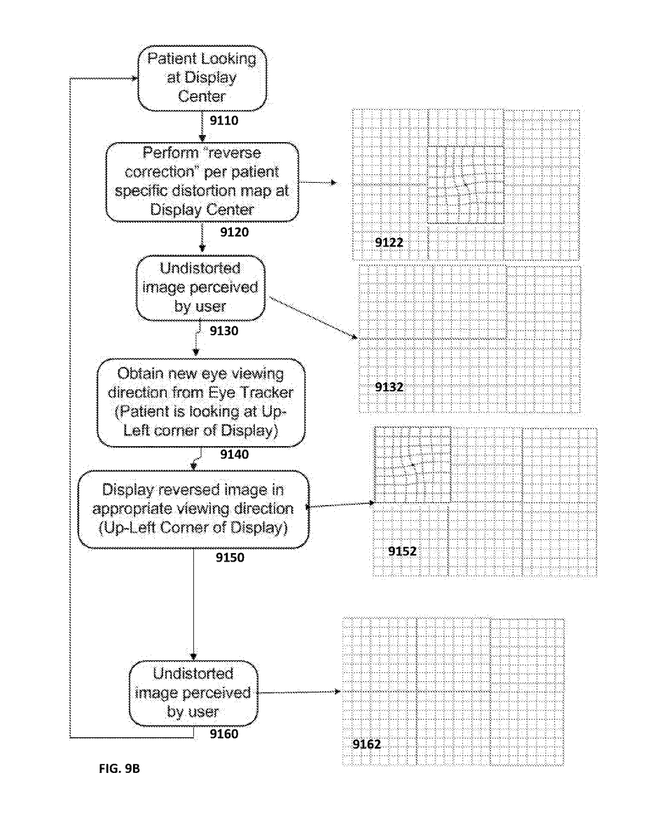

In some additional examples, the technique of the invention may be used for distorted image correction as exemplified in FIGS. 9A and 9B. FIG. 9A exemplifies reverse distortion correction and FIG. 9B exemplifies moving reverse distortion in accordance with user's ROI.

Generally, as shown in FIG. 9A, according to the present technique data about distortion mapping 9012 of the user's vision may be determined by a professional to provide accurate data about vision requirement. According to the present technique, providing corrected display includes selection of data 9010 to be displayed 9011. The local image processing actions 9020 may include applying reverse correction in accordance with the predetermined distortion mapping 9012 to provide corrected and reversed image data 9022. The line of sight of the user is determined 9030 to select the corresponding ROI and display 9040 the corrected image 9042 on the display device. In response to the image data with reverse distortion 9042, the user may perceive the displayed image(s) 9050 in normal or improved fashion 9052 enabling improved vision to the user.

Further, as shown in FIG. 9B, the technique includes alignment of the corrected display data in accordance with user's ROI. As shown, the technique includes providing display content 9110, and correction of the display data in accordance with vision requirements and region of interest of the user. Having determined the region of interest as described above, the image processing actions may typically include performing of reverse distortion 9120 based on the known vision distortion of the user. An example of reverse distortion is shown in 9122. The corrected display data is transmitted to the display device 9130 and enables the user to view non-distorted image 9132. Further, when the user's line of sight or region of interest varies, the technique flows and determined new ROI for image processing 9140. The reverse distortion is applied to the display data 9150 in the new location 9152 to continue and provide the user with improved image 9160 forming undistorted image on the user's retina 9162.

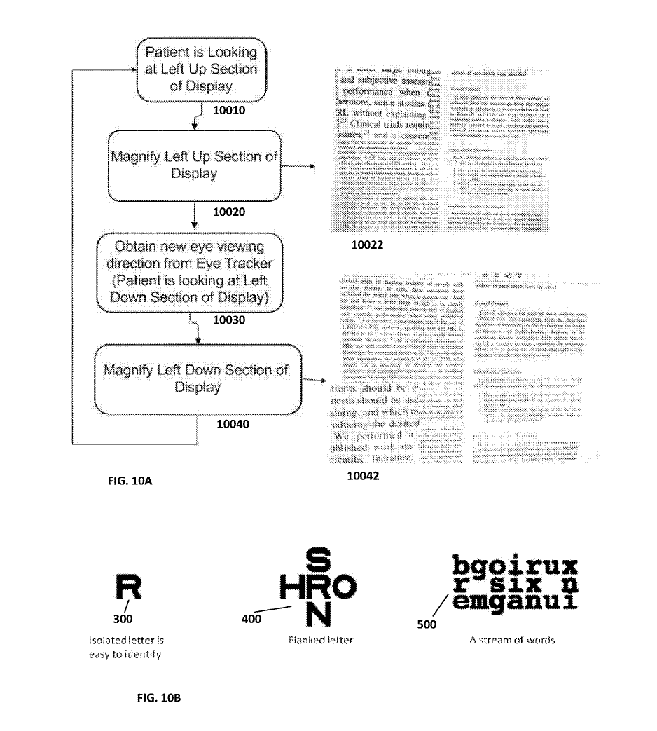

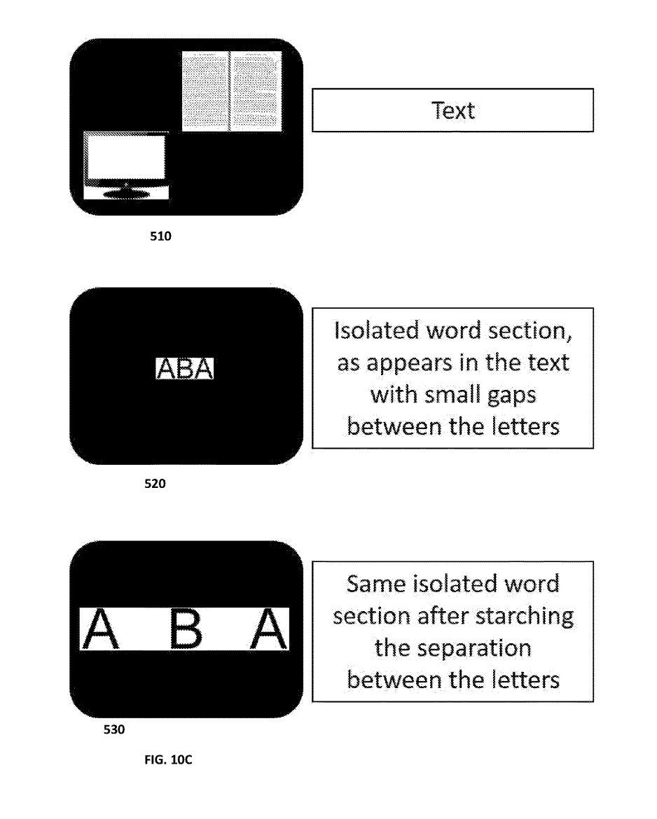

Reference is made to FIGS. 10A and 10B exemplifying image correction associated with textual data for improving reading ability for users. FIG. 10A exemplifies magnification of portions of text data and FIG. 10B exemplifies isolation of textual data to prevent crowding.

As shown in FIG. 10A, when textual data is presented to the user, the technique includes determining a ROI within the data 10010. In this example, when the user shifts his region of interest to a different location (e.g. top/left) of the display, the image processing action selected in magnification of the text data 10020 to provide enlarged text in the new location of the ROI (shown in the figured on top/left of the display region 10022) Similarly, to the above, when the user varies his region of interest to bottom/left of the display, new location is detected 10030 and the corresponding text is enlarged on bottom/left of the display region 10040 and 10042.

It should be noted that the region of interest maybe as small as a single letter or word or be as large as a paragraph or more. In some configurations, the ROI may follow reading pace of the user and directing the user's attention to the following text to be read. In some additional configurations, the technique may further utilize vocal assistance, e.g. reading out loud of the text within the ROI in a predetermined selected pace.

Additionally or alternatively, the present technique may utilize separation of letters or words within textual data to provide easier reading to user with corresponding vision requirements. This is exemplified in FIGS. 10B to 10D exemplifying differences between isolated letters and textual portions. FIG. 10B exemplifies isolated letter 300, flanked letters 400 that may make is harder for users with vision impediments to read, and word written in tight text 500, which may also make it difficult for users to identify. FIG. 10C exemplifies division of text data to single letter to reduce crowding and FIG. 10D exemplifies isolating of sentences to assist in reading.

As shown in FIG. 10C, textual data 510 may be difficult to read for users having certain vision requirements (vision impairment). In order to reduce crowding, according to some embodiment the present technique may utilize division of continues text (e.g. taken from digital image) divided into small segments 520. For example, a small segment may be a single word, a few words, or even a few letters or a single letter in accordance with vision requirements of the user to allow the user to identify the words easily. In some cases isolating words may not be sufficient for eliminating the crowding effect. Thus, in accordance with certain vision requirements of some users, the technique may further enlarge the separation between the letters within a selected word 530 in the ROI. This is in order to reduce the crowding effect and enable reading to be easier or even possible for the user.

An alternative processing is exemplified in FIG. 10D where the technique utilizes isolation of sentences within text to provide reading assistance. From a page of textual data 610, a paragraph 620 or even single sentence 630 may be isolated to direct the user's eyes to the text being read at the time and reduce distractions. Generally, several techniques may be used for text isolation. For example, surrounding text may be blurred around the selected text; the surrounding text may be removed from the display data leaving black spaced in white, gray or background color; the selected section of the text may be highlighted with selected color different than the surrounding; and the selected text may be enlarged with respect to the text around it, which may be presented in smaller size.

Generally, the selected text may be replaced in accordance with reading progress to provide the user with continuous reading experience. Text selection may be based on line of sight of the user, manually (e.g. using keyboard or mouse) or automatically. For example, when the user finishes reading the currently selected/highlighted text the next word/line will be highlighted instead of the previous one.

In order to reduce the crowding effect, the technique of the invention may operate to analyze the ROI and the text therein and perform suitable image processing actions on the text. Generally, the output corrected text may be isolated letters, words or sentences with greater vertical separation. Generally, the output includes at least a portion of the text with enlarged separation between the letters. The output may also be enlarged or not and may include image brightness or contrast variation as the case may be.

The technique may utilize the actual recorded image and location of the gaps between the letters, making the letters larger, or use text recognition algorithm or operate on text data. The text is typically rebuilt in a less crowded way to be presented to the user. The separation between the letters may typically be adjustable.

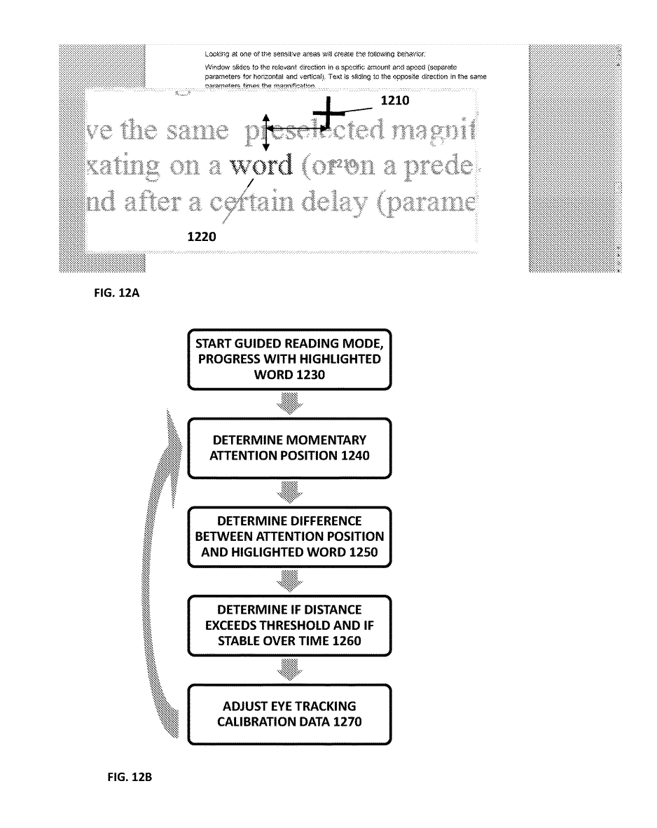

The technique of the invention may also be used for reading assistance as described above utilizing several option techniques including: letting the ROI follow the user attention directly; imposing relatively constant reading pace on ROI movement; or by a combination of the two. In some configuration the ROI is determined by following the user attention at all times, thus constant movement and especially unwanted movements of the eyes, which are considered as of low relevance to the reading pace, may make the reading difficult. In this case a preferable embodiment of the invention utilizes determining a static region at the center of the ROI in which attention of the user creates no movement, and determining one or more sensitive or command related regions at the periphery of the ROI, such that when the line of sight of the user is identified to be within one of the command regions, the ROI shifts in accordance with the suitable command Alternatively, when constant pace is imposed on the ROI movement or a combination of user attention and constant pace are used, the ROI may typically be moved at a constant speed along a preselected paragraph, and the attention of the user (line of sight) may be used to stop or resume the constant movement and also to allow for automatic adjustment of the reading pace. This operational configuration of the system or technique of the present invention may also be used for reading training, e.g. by receiving vocal input associated with the user reading words out-loud and following the user's propagation accordingly. Generally, one or more words within the text are marked and followed based on user's ROI as exemplified in FIG. 12A, as well as based on predetermined selected pace for reading. In some embodiments, the read out words may be analyzed with voice recognition and compared to the written text in order to provide the user with reading guiding, training, and feedback.

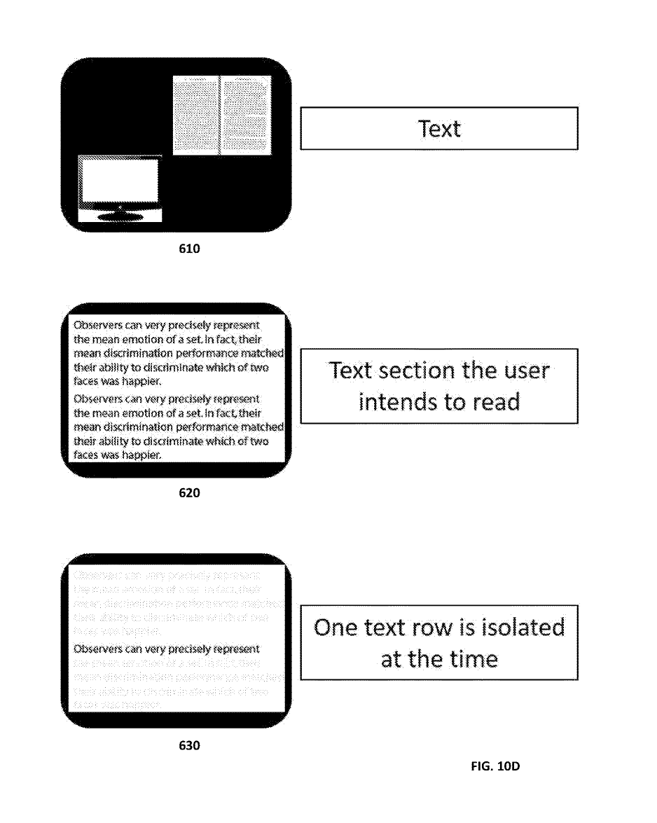

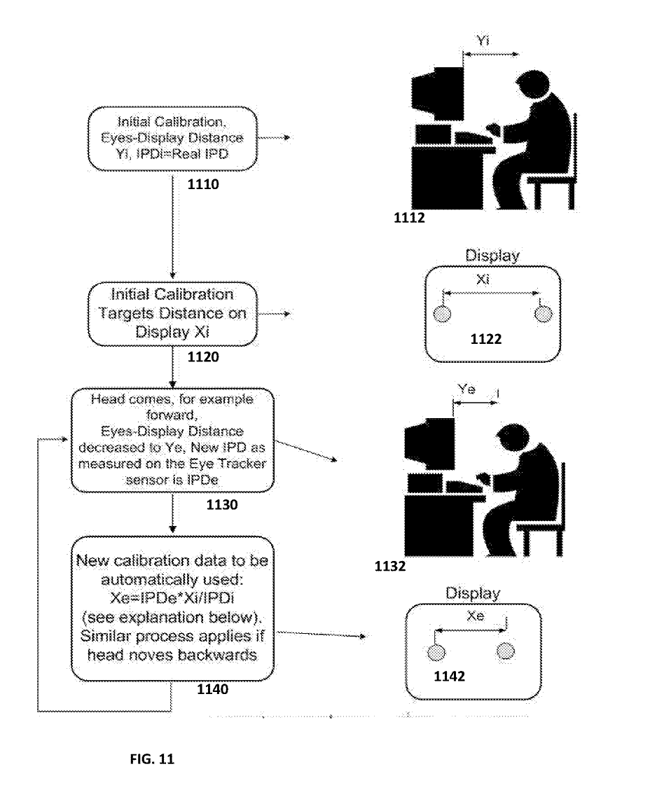

Generally, eye tracking units and devices may require periodic calibration process. However, the technique of the present invention, utilizing line of sight detection in combination with analysis of the data presented to the user may operate to perform the periodic calibration while requiring no additional input or special user operation. More specifically, when operating in guided reading mode, i.e. automatic propagation of marked text while user is reading or adjusting the ROI based on user's line of sight, provides indications of correlation between user's eye orientation and line of sight with respect to the display device. This is exemplified in FIG. 11 showing steps of eye tracking working distance error compensation in a way of a block diagram. As shown, initial calibration is typically provided 1110 when the user is in front of the display device 1112, the initial calibration may also be simply the previous calibration data used. The initial calibration indicates two or more locations on the display device 1122 in accordance with corresponding two or more angular orientations of the user's eyes 1120. Therefore, even if the user changes his head location 1130, e.g. distance to the display device 1132, new calibration data can be determined in accordance with trigonometry data 1140 to determine a shift between points on the display 1142, which may vary due to head movement.

The use of guided reading as exemplified in FIG. 12A above may also be used for continuous calibration of the eye tracking. This is further exemplified in FIGS. 12A and 12B exemplifying the use of guided reading configuration with eye tracking for calibration of the eye tracking data. FIG. 12A exemplifies deviation of line of sight from selected word or element and FIG. 12B exemplifies a flow chart of calibration data adjustment in guided reading.

As shown in FIG. 12A, a highlighted word 1220, “word” in this example is marked by blurring the surrounding text or by any other method. The line of sight 1210 of the user may be determined at a certain location of the display data, typically within certain distance along the horizontal and vertical directions from the selected word 1220. The flow chart in FIG. 12B exemplifies this technique. As guided reading mode is started 1230 and the selected word(s) is(are) highlighted, the technique include determining and following line of sight of the user 1240. The technique may further include determining a distance between the line of sight and the selected word along horizontal and vertical axes 1250 and verify if the distance exceeds a predetermined corresponding threshold 1260. If the distance exceeds the threshold and being stable along a predefined period of time, eye tracking calibration data is updated 1270 to correct the determination of the line of sight. It should be noted that the threshold may be determined in accordance with user data such as vision requirement and the time the user is willing to follow guided reading. This is as users may shift their attention from the text while reading, and thus the distance between the line of sight and the selected text may increase naturally.

Thus, the present invention provides a technique for use on aiding vision for users based on known vision requirements. The technique may be implemented in a computer device, being stationary or mobile (e.g. mobile phone, laptop etc.) or in any dedicated system. It should also be understood that the method and system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention. Also, as indicated above, the technique may utilize any type of display device, being integral with the system of the invention or not. And providing simple 2D image data or capable of presenting separate image data to each of the user's eyes to provide 3D-like experience. The display device may be head-mounted (e.g. glasses) or any other type such as television, computer screen, projectors configured for projecting on a selected surface etc. Those skilled in the art will readily appreciate that various modifications and changes can be applied to the embodiments of the invention as hereinbefore described without departing from its scope defined in and by the appended claims.

1-41. (canceled)

42. A method for personalizing display of data on a display device to a vision requirement of a user, the method comprising:
(a) receiving input about a vision requirement of a user;
(b) generating initial display data;
(c) providing said initial display data to be displayed on said display device;
(d) while said initial display data is displayed on said display device, identifying a region of interest of said user within the initial display data;
(e) based on said identified region of interest and said input about said vision requirement, generating refreshed display data including a portion of the initial display data within the region of interest having undergone image processing to be suited to said vision requirement; and
(f) providing said refreshed display data to be displayed on said display device, thereby to display to said user an image suited to said vision requirement within said region of interest.

43. The method of claim 42, wherein said identifying said region of interest of the user comprises obtaining data about a line of sight of at least one eye of said user and identifying said region of interest based on said line of sight.

44. The method of claim 43, wherein said obtaining data about said line of sight comprises determining a point within a display region of the display device, said point being associated with an estimated location of user attention.

45. The method of claim 43, wherein said obtaining data about said line of sight comprises receiving data about movement history of a pointing device operated by said user and processing said data about movement history for determining said data about said line of sight.

46. The method of claim 44, wherein said obtaining data about said line of sight comprises collecting image data of the user from an imager mounted at a predetermined location with respect to said display device and processing said image data to determine an orientation of at least one of the user's eyes with respect to said display device, thereby to identify said line of sight of said at least one of said user's eyes.

47. The method of claim 46, wherein said obtaining data about said line of sight comprises obtaining data about individual movement of each of the user's eyes and using said data about individual movement to identify a right eye line of sight and a left eye line of sight, and
wherein said initial display data comprises initial right-eye display data and initial left-eye display data, and said generating refreshed display data comprises applying first and second image processing techniques to at least a portion of said initial right-eye display data and said initial left-eye display data, respectively.

48. The method of claim 47, wherein said obtaining data about said individual movement of each of said user's eyes further comprises determining if at least one of said right eye line of sight and said left eye line of sight is outside of a display region of said display device, and while at least one of said right eye line of sight and said left eye line of sight is outside of said display region, postponing said generating refreshed display data or transmitting to said display device a command to darken at least part of said display region.

49. The method of claim 42, wherein said generating said refreshed display data comprises applying to said portion of said initial display data within said region of interest at least one filter in accordance with said input about said vision requirement, said at least one filter selected from the group consisting of: image shift, image rotation, image distortion reversing, image magnification, increase in spacing between portions of the image portions, blurring of a surrounding of said region of interest, emphasis of a portion of said region of interest, isolation of a portion of said region of interest, increase in contrast within said region of interest, decrease in contrast of a surrounding of said region of interest, increase in brightness of said region of interest, replacement of image colors within said region of interest with colors of a preselected color map, removal of defected portions of said initial display data, marking of contours within said initial display data, and marking of pattern edges within said initial display data.

50. The method of claim 42, wherein steps (b)-(f) are carried out repeatedly and continuously, thereby continually improving the image displayed to the user.

51. The method of claim 42, further comprising:
identifying one or more command regions within said initial display data, each of said one or more command regions associated with a predetermined command;
determining whether said region of interest of said user is within said one or more command regions; and
if said region of interest is within said one or more command regions, activating at least one said predetermined command associated with said one or more command regions.

52. The method of claim 42, further comprising:
tracking eye movement of said user;
identifying one or more gestures corresponding to said eye movement of the user, said one or more gestures being associated with one or more predetermined commands; and
in response to identification of said one or more gestures, activating said one or more predetermined commands.

53. The method of claim 42, configured for providing reading assistance, wherein said generating refreshed display data includes identifying one or more data portions to be highlighted and computing a rate of reading progress and highlighting selected portions of said refreshed display data accordingly.

54. A system for personalizing display data, to be displayed on a display device, to a vision requirement of a user, the system comprising:
an input module receiving input about a vision requirement of a user;
an output module outputting display data for display on a display device; and
at least one processing unit, including:
an initial display data generator adapted to receive data to be displayed and to generate initial display data and to provide said initial display data for display on the display device;
a region of interest identifier adapted to identify a region of interest of said user; and
an image processing module adapted to generate refreshed display data based on said region of interest identified by said region of interest identifier and based on said input about said vision requirement, and to provide transmit said refreshed display data for display on said display device.

55. The system of claim 54, wherein said region of interest identifier is functionally associated with a line of sight detector adapted to identify a line of sight of the user, said region of interest identifier adapted to identify said region of interest based on data about said line of sight received from said line of sight detector.

56. The system of claim 55, wherein said line of sight detector is adapted to track movement history of a pointing device connected to said input module, thereby to detect said line of sight.

57. The system of claim 55, wherein said line of sight detector is functionally associated with an eye tracking unit and is adapted to receive from said eye tracking unit input about at least one of location and orientation of at least one of the user's eyes.

58. The system of claim 57, wherein said line of sight detector is adapted to receive from said eye tracking unit input about individual movement of each of the user's eyes, and to identify a right eye line of sight and a left eye line of sight based on said input about individual movement.

59. The system of claim 55, wherein said line of sight detector is further adapted to identify whether said line of sight is within a region of display of said display device and to generate a corresponding notification to the region of interest identifier.

60. The system of claim 55, wherein said image processing module is adapted to apply to said portion of said initial display data within said region of interest at least one filter in accordance with said input about said vision requirement, said at least one filter selected from the group consisting of: image shift, image rotation, image distortion reversing, image magnification, increase in spacing between portions of the image portions, blurring of a surrounding of said region of interest, emphasis of a portion of said region of interest, isolation of a portion of said region of interest, increase in contrast within said region of interest, decrease in contrast of a surrounding of said region of interest, increase in brightness of said region of interest, replacement of image colors within said region of interest with colors of a preselected color map, removal of defected portions of said initial display data, marking of contours within said initial display data, and marking of pattern edges within said initial display data.

61. The system of claim 54, wherein said at least one processing unit further comprises a command generator adapted to identify one or more command regions within the display data and, responsive to a line of sight of said user being detected within said one or more command regions, to generate a corresponding one or more predetermined commands.
